(19) United States
(12) Patent Application Publication
(10) Pub. No.: US 2026/0263488 A1
(43) Pub. Date: Sep. 10, 2026

(54) DELIVERY OF A COOLING AGENT TO THE PHARYNGEAL-ESOPHAGEAL SURFACE

(71) Applicant: Edward Tak Wei, BERKELEY, CA (US)

(72) Inventor: Edward Tak Wei, BERKELEY, CA (US)

(21) Appl. No.: 19/666,335

(22) Filed: May 3, 2026

Related U.S. Application Data
(63) Continuation-in-part of application No. 17/803,407, filed on Jun. 23, 2022, now abandoned.
Application No. 17/803,407 is a continuation-in-part of application No. 16/601,056, filed on Oct. 14, 2019, now abandoned.

(51) Int. Cl. A61K 31/66; A61K 31/221; A61P 1/00

(52) U.S. Cl. CPC A61K 31/66; A61K 31/221; A61P 1/00

(57) ABSTRACT

Disclosed are methods and compositions for treating symptoms of esophageal disorders by localized delivery of a cooling agent to pharyngeal and upper esophageal surfaces. A solid medicament comprising a cooling agent incorporated into a gelatin-based matrix is orally administered. The medicament has a non-spherical geometry for temporary retention within anatomical recesses of the pharynx prior to passage into the esophagus. The medicament has a mass and geometry selected for prolonged pharyngeal residence while permitting spontaneous swallowing.

### CROSS-REFERENCE TO RELATED APPLICATIONS

This application is a continuation-in-part of U.S. application Ser. No. 17/803,407 filed Jun. 23, 2022, which is incorporated herein by reference in its entirety.

### BACKGROUND OF THE INVENTION

No currently available topical formulation is designed to directly modulate sensory nerve endings of the esophageal surface for treatment of esophageal discomfort.

The primary therapeutic target of this discovery is sensory nerve endings of the upper esophageal mucosa, with secondary modulation of contiguous hypopharyngeal afferents.

The lumen of the mouth the esophagus and not into the airway. The efficiency of this system is visible and self-, pharynx, and esophagus is a conduit for food and liquid. On an average day for an adult, this conduit contacts approximately 2 L of fluids, 1 L of saliva, and 2 kg of food. Traffic flow in the conduit is synchronized to ensure that food particles and liquids travel down evident, for example, when a large meal is consumed with a drink. The masticated bolus transit from mouth to stomach and swallowing occurs in about one second. When the bolus is pushed into the oropharynx and swallowed, the epiglottis, like a trapdoor, drops over the glottis. The pharynx connects to the proximal end of the esophagus at the upper esophageal sphincter. The distal segment of the esophagus terminates at the lower esophageal sphincter, at its junction with the stomach.

The lumenal surfaces of the pharynx and esophagus contact physical, chemical, and biological agents such as refluxed digestive enzymes and acid, food particles, infectious agents such as viruses, and mediators of the immune response. Symptoms of inflammation in this foodway manifest as dysphagia, indigestion (dyspepsia), satiety, nausea, globus, epigastric pain, heartburn, chest pain, regurgitation, a sour taste in the mouth, eructation (belching), and sometimes halitosis and hiccups. These sensory expression of discomfort feel different from irritation of other body parts, such as the skin. The receptive fields of nerve endings for noxious signals in the esophagus are mainly spinal afferents and some afferents of the 9th, 10th cranial nerves. These nerves also convey signals of thermosensation, such as cooling.

A variety of agents are used to treat the discomforts of acid reflux and indigestion. These include proton-pump inhibitors such as omeprazole and H2-receptor antagonists such as ranitidine. None of these preparations contain a cooling agent designed to affect sensory nerve endings in the upper eosphagus. About four decades ago, scientists at Wilkinson Sword (WS) synthesized over 1200 compounds to find cooling agents that had cooling properties [New compounds with the menthol cooling effect. J. Soc. Cosmet. Chem. 29:185-200, 1978]. From this research, N-alkyl-cycloalkyl- and an N-alkyl-alkyl carboxamide such as WS-3, WS-5, WS-12, and WS-23, were identified and are used today as additives for confectionery, comestibles, (e.g., chewing gum), toothpaste and toiletries. These compounds are not used for medical treatment of indigestion.

Topical drug delivery to the esophageal lining is a formidable technical challenge. The anatomy of this part of the aerodigestive system is complex (FIG. 1). This subject has been extensively discussed in the context of seeking treatments for eosinophilic esophagitis (EE), an immune disorder that affects children [Hirano et al., Drug Development for Eosinophilic Esophagitis. Clin Gastroenterol Hepatol. 2017. 15:1173-1183. doi: 10.1016/j.cgh.2017.03.016; Krause et al., The EsoCap-system. An innovative platform to drug targeting in the esophagus. J Control Release 2020:327:1-7. doi.org/10.1016/j.jconrel.2020.08.011]. The standard treatment for acid reflux disorders such as proton-pump inhibitors (PPI) do not work for EE, so the goals are to have systems for delivering anti-inflammatory steroids by topical administration. The main hindrance to topical esophageal drug delivery is the fast transit time (≤1 sec) of any materials that enter the esophagus.

In summary, the invention provides a method and formulation for delivering a cooling agent by localized, topical application to the hypopharynx and upper esophagus. The delivery system is a non-spherical, gelatinous Shaped-Gel that is configured to be temporarily intercepted and retained within luminal recesses of the hypopharynx, including the valleculae and pyriform sinuses, before progressing into the esophagus. This deliberate delay in pharyngeal transit increases residence time at the target surfaces and allows dissolution of the Shaped-Gel in saliva, thereby enabling effective delivery of the active ingredient to sensory receptors of the hypopharynx and upper esophagus.

Based on the inventor's prior direct experience and human testing, a class of cooling compounds known as 1-dialkyl-phosphinoyl-alkanes (DIPA) was selected and evaluated, and preferred embodiments were identified as having sensory properties suitable for therapeutic use. Selected DIPA compounds produce a comfortable, sustained cooling sensation perceived at anatomical landmarks corresponding to the Adam's apple, jugular notch, and manubrium, indicating delivery to the intended esophageal regions. The centrally perceived cooling sensation alleviates sensory discomfort originating from the esophagus and is effective in relieving symptoms including heartburn, epigastric pain, regurgitation-associated discomfort, sensations of bloating or indigestion, dyspepsia, and belching.

In preferred embodiments, the DIPA compound is formulated in a Shaped-Gel matrix having a mass of approximately 0.5 to 1.0 g and a generally flat geometry that facilitates swallowing while promoting temporary pharyngeal retention. The amount of DIPA per Shaped-Gel is preferably in the range of approximately 3 to 15 mg and is effective for reducing sensory discomfort of the esophageal tract in a subject in need of treatment.

### SUMMARY OF THE INVENTION

Disorders of the esophagus and adjacent pharyngeal structures produce symptoms including heartburn, chest discomfort, dyspepsia, and globus sensation. These symptoms are mediated in part by sensory nerve endings distributed throughout the hypopharynx and upper esophagus.

Despite the prevalence of these conditions, no topical therapy is designed to deliver a sensory modulating agent to the pharyngeal esophageal surface in a controlled and localized manner. Current treatments primarily alter gastric acidity or systemic physiology and do not directly address sensory irritation at the esophageal surface.

Topical delivery of therapeutic agents to the esophageal lining presents a unique technical challenge due to the extremely short transit time of swallowed materials, typically less than one second (FIG. 2). Conventional oral dosage forms are designed to traverse the pharynx and esophagus rapidly, limiting contact between the active agent and esophageal tissue. The problem of drug delivery to the upper esophageal lining is inherent in the anatomy and physiology of the delivery sites. The upper esophageal sphincter limits access to the esophageal lumen, but when the sphincter opens, it is sudden and the bolus is past the upper esophagus is ≤1 sec. There is no time for contents of the swallowed bolus to contact the esophageal lining.

The invention's novelty lies in the discovery that the active agent, embedded in a gel matrix, can be temporarily retained in the pyriform sinuses and valleculae, from which it is subsequently dissolved by saliva and slowly delivered to the upper esophagus. This detour allows the active ingredient to reach targets that would otherwise be inaccessible within the constraints of normal swallowing physiology (FIG. 2).

The applicant provides the invention's Core discovery: A Purposeful Anatomical Detour. The inventive concept is a delivery system that intentionally prolongs retention of a medicament in the pharyngeal region, thereby increasing effective exposure of the upper esophageal surface to a topical therapeutic agent.

In one aspect of the present invention a therapeutic method is described to treat symptoms of an esosphagus disorder whereby a medicament herein often referred to as a “Shaped-Gel” is orally administered in a gelatin-glycerol-water matrix. The Shaped-Gel contains a cooling agent which is selected from a 1-dialkyl-phosphinoyl-alkane, a p-menthanecarboxamide, or a diisopropylbutanamide. The Shaped Gel is configured with a non-spherical geometry and dimensional proportions selected to delay passage through the pharynx while permitting spontaneous swallowing without discomfort. The optimal shapes comprise a size of ~0.5 to 1 g, a non-ellipsoid or spherical shape, a shape with increased surface area, and three-dimensional axes in which one axes was 10-30% the length of the other two axes. Preferred shapes were a pentagon or a hexagon, a square, or most preferably a torus. The most likely trapping loci of the Shaped-Gel or its fragments are identified as the valleculae and pyriform sinuses of the oropharynx and hypopharynx, respectively.

The active ingredient is formulated in a gelatin, glycerol, and water matrix such that it will quickly dissolve in body fluids such as saliva. Solubility in bodily fluids ensures safety because entry of the gel into airways will dissolve and not obstruct. This gel is called a “Shaped-Gel,” and its size and shape are configured to be intercepted, impeded, and retained in the pharyngeal valleculae and pyriform sinuses before it passes down the esophagus. The non-standard shape prolongs transit time of the Shaped-Gel and allows the active ingredient greater contact time with its receptor.

The selected compound has a sensory effect on the esophagus that evokes coolness and counteracts discomfort (heartburn, sour taste, and pain). This sensation is similar to when ice cream is swallowed but lasts longer. The choice of a particular cooling molecule is based on the applicants past studies of cooling agents. The favored class are compounds known as phosphine oxides (Cryosims). Other classes are p-menthane carboxamides, and di-isopropylbutylamides. The features of the preferred embodiment are molecular size (preferably ≤300 Daltons), water solubility (≥1 mg/ml of water), a cooling sensation without cold discomfort at the therapeutically effective dose, sufficient potency (≤20 mg per single dose) and sufficient duration of cooling action after a single dose ≥30 min.

The inventive step here is to do the opposite of what is conventional and expected. Normally, a swallowed tablet is designed to move quickly into the stomach. Here, the design is a “Shaped-Gel” configuration (FIG. 3) that will take a detour and be retained in the pharyngeal valleculae and pyriform sinuses before it passes down the esophagus. This design is counterintuitive because the golden rule is to avoid pharyngeal residue, not create residue.

The goal is prolonging the transit time of the Shaped-Gel so the active ingredient has more time to dissolve in saliva, disperse, and reach receptors for cooling (FIG. 2). By experiment, an ideal formulation of Shaped-Gel was a torus or a flat rectangle, with a mass of about 0.5 to 0.8 g (FIG. 3). These shapes increase the surface area to volume ratio, thus enhancing dissolution. Flatness was defined as a pill with the shortest axis, preferentially 5 to 30% of the longest axis. A hemi-torus also worked well. In addition, the presence of single or multiple holes in the Shaped-Gel may enhance the available surface area for dissolution. The Shaped-Gel itself was composed of gelatin, glycerol, and water. Gelatin may be replaced by other gelatinous substances and glycerol by other plasticizers (vide infra). The Shaped-Gel does not affect taste buds on the tongue surface. Also, the Shaped-Gel, in contrast to liquid drops, allows the administration of a higher dose of the active ingredient.

In summary, the proposed design of a Shaped-Gel method of drug delivery relies on a shape that is intercepted in the valleculae and pyriform sinuses above the upper esophageal sphincter. The delay in transit gives the Shaped-Gel cooling ingredient more time to dissolve in saliva and act on the hypopharynx and the esophagus receptors. The cooling agent in Shaped-Gel alleviates symptoms of pharyngitis, esophagitis, reflux disease, chest pain, and dyspepsia are relieved.

### BRIEF DESCRIPTION OF DRAWINGS

FIG. 1 is a diagram of the upper digestive tract and some of its connections.

FIG. 2 is a diagram of the upper digestive tract illustrating the transit of a swallowed medicament. The delivery site is at the back of the mouth. The address of the target for delivery of the cooling agent is the upper third of the esophagus, below the esophageal sphincter. If the medicament is spherical the pharyngeal transit time (PTT) is ≤1 sec. But the medicament is a shaped-gel, such as a torus, it may be retained in the pharynx for ~30 to 60 sec before dissolution. Sites for retention are the valleculae or pyriform sinuses.

FIG. 3 shows a Representative Shaped-Gel geometries. Theses non-spherical Shaped-Gel configurations (torus, pentagon, and square) have increased surface-area-to-volume ratios relative to a sphere of equal mass.

### DETAILED DESCRIPTION OF THE INVENTION

The present discovery pertains to formulations comprising a cooling agent for localized, topical application to sensory nerve endings of the upper esophagus. The formulation is useful for treating, including selectively suppressing, sensory discomfort associated with disorders of the esophageal tract. The onset of the therapeutic effect is rapid (≤5 minutes) and of sufficient duration to provide clinical benefit. At present, there are no commercially available products that operate via this mechanism of action. Accordingly, the disclosed formulation and its selected cooling agents may be useful for treating conditions including, but not limited to, esophageal discomfort, cough, esophagitis, heartburn, regurgitation associated discomfort, dysphagia, dyspnea, dyspepsia, chest pain, and acid reflux-associated discomfort.

The inventive step includes a gel coated tablet that is intentionally shaped to be retained in the throat following swallowing. Conventional gelatin coated pills containing liquid or semi liquid contents are designed primarily to facilitate swallowability and rapid transit from the oral cavity to the stomach. Standard softgel pills are typically spherical, oblong, oval, elliptical, or almond shaped and are configured to pass quickly through the pharynx. An accompanying sip of water reduces friction and increases bolus volume, allowing the gel to glide past the upper esophageal sphincter with a pharyngeal transit time (PTT) of ≤1 second. Such gel capsules are further designed to minimize pharyngeal residue, as measured by videofluoroscopy. The gelatin shell prevents dispersion of the capsule contents within the oral cavity, thereby avoiding stimulation of taste buds and preventing an unpleasant sticky or chalky mouthfeel.

In contrast, the inventive “Shaped Gel” refers to a gelatin or gelatin like coated object that is deliberately configured for deposition, temporary retention, and rapid dissolution within the pharynx. Upon dissolution, the contents are released onto the surfaces of the hypopharynx, the upper esophageal sphincter, and the upper esophagus. The formulation comprises a cooling agent, gelatin, a plasticizer such as glycerol or sorbitol, and water. These components are warmed to form a liquid solution and subsequently cooled within a mold to produce a three-dimensional object. The three-dimensional configuration of the Shaped Gel is selected to promote targeted delivery of the cooling agent to the pharynx and, subsequently, to the esophagus.

Preferably, the Shaped Gel is generally flat, having a short axis measuring approximately 5% to 45% of the longest axis. The object preferably exhibits a high surface area to volume ratio and a mass between 0.5 and 1.0 g, and more preferably between 0.4 and 0.6 g. This configuration facilitates interception and retention of the object within anatomical recesses of the pharynx, including the valleculae and pyriform sinuses. The resulting delay in esophageal passage allows saliva mediated dissolution of the active ingredient and its subsequent distribution to target sensory receptors in the upper esophagus.

Dissolution of the Shaped Gel cannot be readily monitored using videofluoroscopy or ultrasound because gelatin is not radiopaque and does not significantly reflect acoustic signals. Accordingly, external anatomical landmarks are used as indirect indicators of the location of the Shaped Gel and the progression of its cooling effect. A subjective sensation of a “lump” posterior to the laryngeal prominence indicates the presence of the Shaped Gel at the level of the pharynx and may be timed using a stopwatch. Cooling perceived at the jugular notch, a readily identifiable external landmark, confirms that the cooling agent has passed below the upper esophageal sphincter. Cooling perceived posterior to the manubrium indicates delivery of the cooling agent to the upper third of the esophagus.

A preferred onset of cooling is ≤5 minutes, and a preferred duration of action is approximately one hour or longer. These parameters may be adjusted, for example, by modifying the gelatin-to-glycerol ratio and/or the water content of the Shaped-Gel. By way of example, increasing the gelatin content from 12% to 15% delayed the onset of cooling from approximately 2 minutes to approximately 8 minutes. Selection of a cooling active ingredient for use in the Shaped-Gel is informed by the applicant's prior experience with cooling agents tested in human subjects, as described in Yang, ET Wei et al., A novel TRPM8 agonist relieves dry eye discomfort, BMC Ophthalmology (2017) 17:101; Lee, ET Wei et al., (2024). Histamine- and pruritogen-induced itch is inhibited by a TRPM8 agonist: a randomized vehicle-controlled human trial. Brit. J. Dermatol. 190 (6), 885-894, https://doi.org/10.1093/bjd/ljae054, and ET Wei (2013), Ser. No. 13/065,971, U.S. Pat. No. 8,426,463, incorporated herein by reference. An alternative preliminary screening criterion for cooling activity is the EC₅₀ of a compound at the TRPM8 receptor as measured in transfected cells; however, this metric has been found to be unreliable when used alone.

Preferably, the selected cooling agent exhibits effective potency in the range of approximately 0.4% to 1.5% (4 mg/ml to 15 mg/mL) within the Shaped-Gel. For a Shaped-Gel having a mass of 1 g, this corresponds to a dose of approximately 4 to 15 mg per unit. In one preferred embodiment, the cooling agent is a nonyl-substituted DIPA compound. Other cooling agents belonging to the 1-dialkylphosphorylalkane and p-menthane carboxamide families may also be used. As will be appreciated by those of ordinary skill in the art, features and preferred embodiments described with respect to one aspect of the discovery may be applied to other aspects of the discovery.

In contrast to the present invention, prior art gelatin drug delivery systems are designed to enhance swallowability and promote rapid transit through the throat, rather than intentional retention within the pharynx. The gelatin-glycerol-water object described herein is therefore referred to as a “Shaped-Gel” to distinguish it from conventional softgels designed for rapid passage.

### Background Definitions and Anatomical Context

### Adam's Apple (Laryngeal Prominence)

The Adam's apple, also known as the laryngeal prominence, is a visible protrusion on the anterior aspect of the human neck formed by the angle of the thyroid cartilage surrounding the larynx. The laryngeal prominence is generally more pronounced in males than in females due to differences in cartilage growth during puberty.

### Cognitive Fields for Coolness

The perception of coolness varies substantially across different regions of the body. Certain areas, including the ocular rim, eye surface, and scrotum, exhibit particularly high sensitivity to cold stimuli. Other sensitive areas include the tip of the nose, anogenital region, neck, flexures of the elbows and knees, and the skin overlying the shoulder blades and ankles. Cooling sensations arising from keratinized skin surfaces are typically localized and spatially discrete.

In contrast, cooling sensations arising from tissues covered by mucous membranes-such as the oropharynx, nasal cavity, and nasopharynx—are perceived as more intense than those arising from the oral cavity. The sensitivity of the hypopharynx and esophagus has received comparatively little scientific attention. However, when cool sensations are perceived within the throat or upper esophageal lumen, individuals often experience a rapid and generalized sensation of whole-body cooling. Cooling at this anatomical location appears to be integrated by the central nervous system as a change in core temperature rather than as a localized surface sensation.

### Cold Discomfort

The term cold discomfort refers to unpleasant sensations including “icy cold,” substernal coldness, or systemic coldness. Icy cold may be perceived within the throat and esophagus and can be acutely painful. Coldness localized to the chest is typically perceived posterior to the sternum and may be similarly uncomfortable. Systemic coldness, comparable to chills, is often first perceived around the eyes and subsequently at the skin over the shoulder blades and ankles. Consideration of cold discomfort limits the selection and dosing of cooling agents intended for safe and localized application to the pharynx and upper esophagus.

### DIPA Compounds

DIPA refers to the class of compounds abbreviated as 1-[diisopropyl-phosphinoyl]-alkanes. The numerical designation associated with a DIPA compound refers to the length of the third alkyl group in the molecule. For example, numeric values of 4 through 10 correspond to butyl, pentyl, hexyl, heptyl, octyl, nonyl, and decyl side chains, respectively. These alkyl groups are linear (“normal” or n-configuration), with the phosphinoyl group attached at the primary (1−) carbon of the third alkyl chain.

Alternative nomenclature for this class includes trialkyl phosphine oxides and 1-dialkylphosphorylalkanes. The compound names derive from phosphinic or phosphoric acid, and the terms “phosphinoyl” and “phosphoryl” are considered equivalent and interchangeable in the context of DIPA compounds.

### Eosinophilic Esophagitis

Eosinophils are a class of white blood cells involved in immune regulation. Under normal conditions, the esophageal mucosa contains few or no eosinophils. A histologic diagnosis of eosinophilic esophagitis (EE) is established when fifteen or more eosinophils per high-power microscopic field are present. Clinical symptoms of EE overlap with gastroesophageal reflux disease (GERD), including heartburn and dysphagia; however, proton pump inhibitors (PPIs) are generally ineffective in EE. Current treatment typically involves topical corticosteroids, though consistent and targeted drug delivery remains challenging. A topical cooling agent delivered to the pharynx or upper esophagus has potential utility in alleviating symptoms associated with EE.

### Epigastric Region

The epigastric region is the central upper abdominal region located above the umbilical region and between the left and right hypochondriac regions. Anatomically, it is a triangular area inferior to the xiphoid process. Epigastric discomfort is a common feature of reflux-related disorders.

### Epiglottic Valleculae

The term vallecula refers to a furrow, depression, or small valley. The epiglottic valleculae are paired anatomical depressions located at the base of the tongue adjacent to the median glossoepiglottic fold. Imaging studies in humans using barium contrast have reported a mean vallecular area of approximately 84 mm² and a mean volume of approximately 1.55 mL. The valleculae can retain saliva and particulate matter and serve as anatomical landmarks during laryngoscopy and endotracheal intubation.

### Esophagus

The esophagus is a muscular tube approximately 25 cm (8-10 inches) in length that connects the hypopharynx to the stomach. It runs posterior to the trachea and anterior to the vertebral column. The esophagus extends from the cricopharyngeus muscle at the level of the cricoid cartilage to the gastric cardia at the level of the diaphragm. In axial cross-section at the neck level, the esophagus occupies a central position within the body and typically has a diameter of approximately 1.5-2 cm.

### Jugular Notch

The jugular notch, also known as the suprasternal notch, is the visible external depression at the center of the neck located between the medial ends of the clavicles and superior to the manubrium of the sternum.

### Liquid Volume to Accompany Swallowing a Shaped-Gel

In clinical pharmacology studies, volumes of approximately 240 ml of water are often administered to facilitate swallowing of oral dosage forms. Smaller volumes, such as 100 mL, have also been used. In one study of 428 participants, the average swallowed volume was approximately 40 mL. In the practice of the present invention, subjects may be instructed to use a sip or teaspoon-sized volume (approximately 5 mL) of liquid, if necessary, to assist swallowing. Swallowed liquid volumes of ≥10 mL promote rapid opening of the upper esophageal sphincter (cricopharyngeus muscle) and reduce pharyngeal transit time.

### Lower Esophageal Sphincter

The lower esophageal sphincter is a specialized segment of the circular smooth muscle layer located at the gastroesophageal junction. Relaxation of this sphincter increases the likelihood of reflux of gastric contents into the esophagus.

### Pharyngeal Anatomy

The pharynx, commonly referred to as the throat, is a funnel-shaped passage connecting the nasal and oral cavities to the esophagus. It is subdivided into the nasopharynx, oropharynx, and hypopharynx (also referred to as the laryngopharynx). The hypopharynx and esophagus occupy central anatomical positions within the neck and upper chest.

At the inferior border of the oropharynx are the epiglottic valleculae, which are paired depressions at the root of the tongue located posterior to the anterior surface of the epiglottis. The oropharynx transitions into the hypopharynx, which in turn connects to the esophagus. Lateral to the larynx within the hypopharynx are the pyriform sinuses. The valleculae and pyriform sinuses form recesses capable of temporarily retaining foreign matter, including irregularly shaped objects.

At the entry to the esophagus lies the upper esophageal sphincter, which is normally closed in a resting state. During swallowing, the sphincter transiently relaxes to permit passage of a swallowed bolus. Pharyngeal transit time is typically ≤1 second in healthy adults.

### Pharyngeal Residues

In up to approximately 20% of elderly individuals, pharyngeal residue may be observed in the valleculae or pyriform sinuses following swallowing. Such residue is detectable using videofluoroscopy with radiopaque contrast agents such as barium sulfate. The pyriform sinuses are a frequent site for particle retention.

### Pharyngeal Transit Time (PTT)

Pharyngeal transit time is defined as the interval between arrival of the bolus tail at the fauces and complete passage of the bolus tail through the upper esophageal sphincter. In healthy adults, average PTT is ≤1 second and is commonly measured using videofluoroscopy.

### Pyriform Recess

On either side of the laryngeal orifice are recesses known as the pyriform recesses (alternatively termed pyriform sinuses or smuggler's fossae). These pear-shaped anatomical spaces are located within the hypopharynx and are known sites for retention of food particles and irregularly shaped objects such as fish bones.

### Receptive Field of a Sensory Neuron

A sensory neuron's receptive field is the spatial region in which a stimulus alters neuronal firing. In epithelial tissues, sensory nerve endings interdigitate near the basal layers of the epithelium. Although an individual receptive field may be small (e.g., on the order of 1 mm²), activation by nociceptive or pruritic stimuli can dominate perceptual awareness.

### Softgel

A softgel is an oral dosage form comprising a gelatin-based shell surrounding a liquid or semi-solid fill. Modern softgels may substitute gelatin with starch or carrageenan. Softgels typically contain gelatin, water, a plasticizer such as glycerol or sorbitol, and an active ingredient. Softgels are designed to promote rapid passage through the pharynx into the stomach and should not be confused with lozenges, pastilles, troches, or hard tablets designed for oral dissolution.

### Softgel Shape and Size

Standard softgel capsules are designed for rapid swallowing and are commonly round, oval, elliptical, or oblong in shape. Large softgels may exceed 0.6-1.2 g and can be difficult for some individuals to swallow. Medicinal tablets generally range from 25 to 325 mg. Tablets with angular geometries are rarely employed due to swallowability concerns.

### Shaped-Gel Geometry

For the practice of the present invention, the Shaped-Gel is preferably flat and configured to be temporarily retained within the valleculae or pyriform sinuses. The shape may be toroidal or polygonal and may include one or more through-holes or cavities to facilitate saliva access and dissolution. Preferred weights range from approximately 0.5 to 1.0 g, more preferably 0.5 to 0.8 g. The preferred geometries possess surface areas at least 133% greater than that of a sphere of equal volume. A flat geometry is defined as having one axis measuring approximately 5%-45%, and preferably 15%-25%, of the longest axis.

### Swallowing (Deglutition)

Swallowing is a complex, highly coordinated neuromuscular process whereby food or liquid is transported from the oral cavity to the stomach. The sensory receptors and motor effectors involved in swallowing have been extensively described in the literature.

### Teaspoon

A teaspoon is a unit of volume commonly used in cooking. Under U.S. FDA regulations (21 CFR § 101.9), one teaspoon equals five milliliters (5 mL).

### Torus and Toroid

In geometry, a torus is a three-dimensional surface generated by revolving a circle around an axis coplanar with the circle. If the generating figure is not a circle, the resulting object is called a toroid. The Shaped-Gel may assume a toroidal or hemitoroidal geometry.

### TRP Channels

Transient receptor potential (TRP) channels are a family of cation channels involved in sensing thermal, nociceptive, and painful stimuli. Their precise physiological and pharmacological roles continue to be elucidated.

### Upper Esophageal Sphincter

The cricopharyngeus muscle forms part of the upper esophageal sphincter mechanism. Located at the junction of the hypopharynx and cervical esophagus at approximately the C5-C6 vertebral level, the sphincter is tonically contracted at rest and relaxes transiently during swallowing to permit passage of a bolus.

### Vallecula

The epiglottic valleculae are paired depressions located at the base of the tongue behind the epiglottis and along the inferior border of the oropharynx. These anatomical recesses may accumulate saliva or particulate matter.

### Videofluoroscopy (VFS)

Videofluoroscopy is a dynamic radiographic technique used to assess swallowing function. During the procedure, the patient swallows a radiopaque contrast medium, such as barium sulfate, while real-time imaging captures bolus movement and pharyngeal dynamics.

### The Shaped-Gel

This invention aims to prepare a Shaped-Gel to retard its transit time and “trap” it in the valleculae and pyriform sinus above the upper esophageal sphincter. The Shaped-Gel dissolves in saliva and its contents disperse in saliva and moves to and adheres to targets on the esophageal lumen. This strategy is counter-intuitive to standard formulations, which aim to improve swallowability and rapid transit to the stomach [Bar-Shalom D et al. Swallowability. Tablets Capsules. 2016; (Jan.): 1-4]. The criteria for implementation are: a) A mass of 0.5 to 1 g, with a preferred mass of 0.6 to 0.8 g. A≤0.5 g Shaped-Gel may transit too quickly past the upper esophageal sphincter and not have time to dissolve. A ≥1 g Shaped-Gel is challenging to swallow for many individuals, b) instructions to swallow the Shaped-Gel with a small volume of liquid (e.g., 2 mL), or sipping from a plastic bottle of water (volumes of 3 to 5 mL). Larger volumes of swallowed liquid will wash the Shaped-Gel too quickly into the lower esophagus, c) a flat shape such that the shortest axis of the object is 5 to 45% the length of the longest axis.

The Shaped-Gel can have cut-outs. e.g., from the center to facilitate the dissolution of the Shaped-Gel in saliva. The cut-out in a toroid Shaped-Gel is equivalent to the “donut hole.” Punch holes can be made in a rectangular prism. A hemitoroid Shaped-Gel also works, especially for trapping in the pyriform sinuses. A pentagon Shaped-Gel was tested, but its shape may be too foreign to patients for general use. The Shaped-Gel is made with a gelatinous matrix such that the final product is soluble in water or saliva. Thus, if the Shaped-Gel is aspirated into the airway, it is still safe to use because it will dissolve and be absorbed. The final product is stable to store and package (e.g. blister packs). The Shaped-Gel may be further adapted for use by special populations, e.g., young children, the elderly, and disabled individuals with difficulties in salivating or swallowing.

In one embodiment, the cooling agent comprises DIPA-1-9 at a concentration of 0.4 to 1.5% wt/wt. In one embodiment, the composition is a Shaped-Gel composition, and comprises DIPA-1-9 at a concentration of 5 to 15 mg/mL. A preferred amount of the compound delivered at the site of the application is 1 to 10 mg. The composition may be provided with suitable packaging and/or in a suitable container. For example, the composition may be in the form of unit oral dosage unit, for example, in a blister pack.

One aspect of the present discovery pertains to 1-dialkyl-phosphinoyl-alkanes (DIPA) for use in a method of treatment (e.g., targeted treatment) of certain disorders (e.g., a diseases), as described herein. In one embodiment, the medicament comprises DIPA-1-9. In one embodiment, the medicament comprises DIPA-1-9 formulated as a Shaped-Gel. Another aspect of the present discovery comprises administering to a patient in need of treatment a therapeutically effective amount of DIPA-1-9, preferably in the form of a pharmaceutical composition. In one embodiment (e.g., of use in methods of therapy, of use in the manufacture of medicaments, of methods of treatment), the treatment is the treatment of esophageal discomfort caused by reflux or inflammation. The term as used herein, relates to unpleasant sensations of heartburn, epigastric pain, regurgitation, hoarseness, cough, dysphagia, bloat and belching.

### Mensuration of Shaped-Gel Geometry

All three-dimensional objects possess a surface area and a volume. For a given mass, a sphere has the minimum possible surface-area-to-volume ratio, whereas non-spherical geometries inherently exhibit higher ratios. In conventional oral dosage forms, spherical or ovoid shapes are favored because they promote rapid swallowing. By contrast, the present disclosure intentionally employs non-spherical geometries to delay pharyngeal transit.

The Shaped-Gel formulations described herein are dimensioned to increase surface area relative to volume while maintaining swallowability. Flattened or annular geometries are particularly effective, as they are readily transported from the tongue into the pharynx yet resist immediate passage into the esophagus during swallowing. Shapes exhibiting a shortened axis relative to other dimensions demonstrate delayed transit compared to spherical or capsule-shaped forms of equivalent mass.

Increased surface area enhances dissolution of the gelatinous matrix in saliva, resulting in controlled release of the cooling agent to sensory nerve endings of the hypopharyngeal and upper esophageal mucosa. Representative geometries include toroidal and hemi-toroidal forms, as well as flattened polygonal shapes such as squares, rectangles, pentagons, and hexagons. Table 1 provides an illustrative comparison of surface-area-to-volume relationships for selected geometries.

In one preferred embodiment, the Shaped-Gel has a mass of from about 0.5 to 0.8 grams and is formed from a gelatin-glycerol-water matrix that dissolves upon contact with saliva. This formulation avoids airway obstruction and permits spontaneous clearance after release of the cooling agent.

The gelling agent may be gelatin, agar, algin, carrageenan, guar gum, gum arabic, locust bean gum, pectin, modified starch, or mixtures thereof. Plasticizers such as glycerol may be included to control flexibility and dissolution properties. Additional pharmaceutically acceptable excipients, including lubricants, sweeteners, flavoring agents, colorants, and release agents, may be incorporated as needed for manufacture and patient acceptability.

### Table 1. Illustrative Comparison of Shaped-Gel Geometries

Table 1 illustrates representative geometries of solid oral dosage forms and their relative surface-area-to-volume characteristics. Numerical values are illustrative and are provided to demonstrate relative trends, not to limit the scope of the disclosure.

TABLE 1
Illustrative Comparison of Shaped-Gel Geometries

| | Formula | | Relative | |
|---|---|---|---|---|
| Shape | for | Formula for | SA/V | Transit |
| (Representative) | Volume | Surface Area | (Illustrative) | Behavior |
| Sphere | $\frac{4}{3} π {r}^{3}$ | 4πr² | Lowest | Rapid swallow |
| Oblong capsule | Approx. | Approx. | Low | Rapid |
| Cube | cylindrical | cylindrical + | Moderate | swallow |
| | a³ | hemispheres | | Intermediate |
| | | 6a² | | |
| Flattened | lwh | 2(lw + lh + | Higher | Delayed |
| rectangle | | wh) | | |
| Torus | 2π²Rr² | 4π²Rr | High | Delayed |
| Hemi-torus | Fractional | Fractional | High | Delayed |

As shown, non-spherical geometries having increased surface-area-to-volume ratios relative to a sphere exhibit delayed pharyngeal transit and enhanced dissolution.

For example, a torus (donut-shape) is a 3-dimensional surface generated by rotating a circle of radius r around an axis within the circle's plane. The distance between the axis and the circle center is known as the major radius (R), whereas the circle radius is called the minor radius (r). The surface area and volume are given by these equations: Surface Area=4πRr, Volume=2π²Rr². For a Shaped-Gel with an R=0.9 cm and an r=0.09 cm, the Surface Area to Volume ratio is 22. For a sphere, the Surface Area to Volume is 3. Thus, dissolution of a toroid Shaped-Gel is more likely than a spherical Shaped-Gel. Planar sides can increase the surface area on the Shaped-Gel, but such shapes may not be patient-friendly. Shaped-Gels shaped to increase surface area and contemplated in this discovery are cones, cubes, cylindrical tanks, rectangular tanks, capsules, caps, conical frustums, pentagons, ellipsoids, and square pyramids.

### Formulation of Shaped-Gel for Treatment

The term “treatment,” as used herein in the context of treating a disorder, refers generally to the treatment of a human or an animal, including veterinary applications, in which a desired therapeutic effect is achieved. Such therapeutic effects include, but are not limited to, inhibition of disease progression (including reduction or cessation of progression), alleviation or reduction of one or more symptoms, amelioration of the disorder, and cure of the disorder. Treatment also encompasses reduction of sensitivity, reduction of hypersensitization, and desensitization phenomena. Prophylactic treatment (i.e., prophylaxis) is also included within the term “treatment,” such as use in subjects who have not yet developed the disorder but are at risk of developing the disorder.

In one embodiment, including use in methods of therapy, use in the manufacture of medicaments, or methods of treatment, the treatment comprises treatment, including selective treatment, of one or more of the following conditions: esophageal tract discomfort; esophageal discomfort; throat irritation; cough; heartburn; chest pain; discomfort associated with regurgitation or a sour or acrid taste in the throat; inflammation of esophageal tissues; and pain of esophageal tissues. In another aspect of treatment, a DIPA compound, including DIPA-1-9, is used to stimulate coolness receptors and generate sensory signals that reduce or prevent dysphagia, sensations of bloating, belching, and hiccups.

Pharmaceutical compositions comprising DIPA compounds, such as DIPA-1-9, may be administered to a subject topically, as described herein. The term “topical application,” as used herein, refers to delivery of the composition onto the surface of the tongue and subsequently to the luminal surfaces of the pharynx and esophagus.

In preferred embodiments, the DIPA compound is formulated as a Shaped-Gel. Additional ingredients may optionally be included, such as preservatives, lubricants, stabilizers, masking agents, coloring agents, and flavoring agents. The formulation may further comprise one or more additional pharmacologically active agents. When formulated as discrete dosage units (for example, vials or individually wrapped units), each unit preferably contains a predetermined amount of the active compound.

Suitable carriers, diluents, excipients, and related formulation components may be selected from those described in standard pharmaceutical references, including Remington's Pharmaceutical Sciences and the Handbook of Pharmaceutical Excipients. The formulations may be prepared using methods well known in the art of pharmacy, including processes in which the active compound is brought into association with one or more pharmaceutically acceptable carriers or accessory ingredients. In general, formulations are prepared by uniformly and intimately combining the active compound with suitable carriers, including liquid carriers, to achieve a homogenous preparation.

The gelling agent employed includes but is not limited to gelatin, agar, algin, carrageenan, guar gum, gum arabic, locust bean gum, pectin, and modified starch, and mixtures thereof. In a preferred embodiment of the present disclosure, the gelling agent used is gelatin. The plasticizer is glycerol as one of the preferred embodiments of the present disclosure. The plasticizer mixes with the gelling agent and a smaller amount of water. A releasing agent such as lecithin, oil, starch, or vegetable oil, may be used to release or lubricate the gel from adhering to its dispensing and manufacturing system. The purpose of the releasing agent is also to help eject the Shaped-Gel from preformed cavities of the blister pack. Other agents, such as sweetening agents (e.g., sucralose, aspartame) or flavoring agents, may be used to enhance the perception of the Shaped-Gel (e.g., coloring agents). Other pharmaceutically acceptable excipients include diluents, disintegrants, binders, surfactants, emulsifiers, and the like.

Gelling agents suitable for use in the Shaped-Gel include, but are not limited to, gelatin, agar, algin, carrageenan, guar gum, gum arabic, locust bean gum, pectin, modified starch, and combinations thereof. In preferred embodiments, the gelling agent is gelatin. The plasticizer is selected from glycerol, sorbitol, or mixtures thereof. In preferred embodiments, the plasticizer is glycerol. The plasticizer is mixed with the gelling agent and an appropriate amount of water to form a gel matrix suitable for molding and dissolution as described herein.

Those of ordinary skill in the art will appreciate that suitable dosages of DIPA compounds, including compositions comprising DIPA-1-9, may vary among subjects. Determination of an optimal dosage generally involves balancing therapeutic benefit with the risk of adverse or deleterious side effects. The selected dosage level depends on a variety of factors, including, but not limited to, the activity of the DIPA compound, route of administration, timing of administration, duration of treatment, use of other drugs or compounds in combination, severity of the disorder, and the species, sex, age, weight, condition, general health, and medical history of the subject. The amount of DIPA compound and route of administration are ultimately determined by the attending physician, veterinarian, or clinician, although dosages are generally selected to achieve effective local concentrations at the site of action without inducing substantial adverse effects.

Administration may be effected in a single dose and is preferably carried out on an as-needed (pro re nata) basis during the course of treatment. Methods for determining effective dosing regimens and modes of administration are well known to those skilled in the art and may vary depending on the formulation used, therapeutic objective, target receptors, and the subject being treated. Single or repeated administrations may be employed, with the dosage level and administration schedule selected by the subject, physician, veterinarian, or clinician.

In preferred embodiments, the dosage of the DIPA compound ranges from approximately 1 mg to 15 mg per dose, with a preferred dosage of approximately 5 mg to 10 mg per Shaped-Gel unit. This corresponds to a concentration of approximately 5 to 15 mg of active ingredient per gram of Shaped-Gel matrix.

### Esophagus and Its Disorders

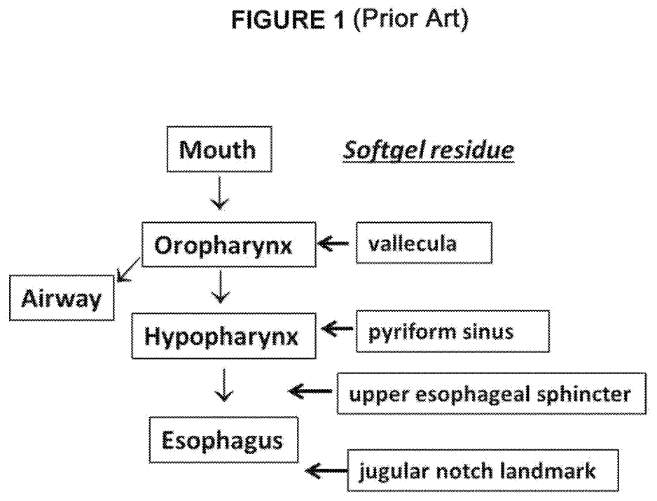

Air enters the body through the nasal cavity and passes into the trachea, whereas food and liquids enter through the mouth, traverse the pharynx, and pass into the esophagus. The esophagus connects the upper esophageal sphincter to the lower esophageal sphincter. In the present application, emphasis is placed on targeting the hypopharynx and the upper third of the esophagus with DIPA-containing Shaped-Gels (FIG. 1).

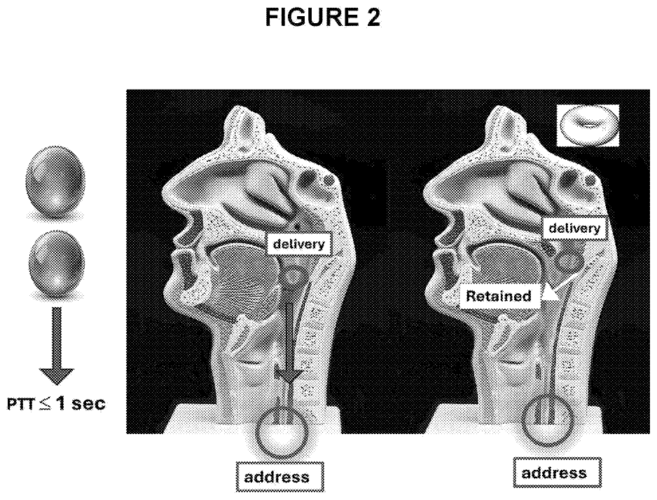

The pharynx is subdivided into the nasopharynx, oropharynx, and hypopharynx, as illustrated in FIG. 2. The oropharynx extends from the soft palate to the level of the hyoid bone, while the hypopharynx (also referred to as the laryngopharynx) extends from the hyoid bone to the cricoid cartilage. The pharyngeal and esophageal luminal surfaces are lined by stratified epithelium, whereas the respiratory tract, including the nasopharynx, larynx, and trachea, is lined by pseudostratified epithelium.

A substantial volume of material passes through the oropharyngeal lumen on a daily basis. An average adult inhales approximately 12,000 L of air per day, ingests roughly 2 L of fluids, secretes approximately 1 L of saliva, and consumes about 2 kg of food. Coordination of these flows is essential for survival, as airflow must be directed to the lungs while food and liquids are directed into the esophagus. Swallowing events occur frequently, often once per minute during waking hours.

The oral cavity contains specialized structures for mastication, salivation, and taste, including the teeth, tongue, and salivary glands. Swallowing a bolus is a complex reflex requiring coordinated activity of multiple cranial nerves and muscle groups. Thermosensation is not a dominant protective reflex in the oral cavity, which tolerates temperatures that would be painful on skin. Cooling sensations, however, play an important role in thirst regulation and are perceived as pleasant in the consumption of cold fluids and foods. Sensory nerves closely monitor temperatures at the oral cavity-pharynx junction, and cooling in this region may rapidly relieve thirst, dryness, and discomfort.

The pharynx contains powerful constrictor muscles arranged to grasp the bolus and propel it into the esophagus. Anatomically, the pharynx narrows significantly at the tongue base and posterior pharyngeal wall, where cross-sectional areas may approach approximately 1 cm², with adjacent mucosal surfaces measuring approximately 3-5 cm². This region, including the valleculae and pyriform sinuses, is a preferred target for drug delivery in the present invention. The Shaped-Gel is designed to lodge temporarily within these recesses, delay pharyngeal transit, dissolve in saliva, and distribute the cooling agent across the mucosal surface to access TRPM8-expressing sensory nerve endings.

Afferent sensory input from the oropharynx, hypopharynx, and upper esophagus is transmitted to the brain via distinct cranial nerves, including the trigeminal (V), glossopharyngeal (IX), and vagus (X) nerves. The brainstem integrates this topographically organized sensory information and coordinates swallowing and esophageal motility. These sensory afferents convey thermosensory and nociceptive information, making them relevant targets for therapeutic modulation using cooling agents.

Chest pain is a common symptom leading patients to seek urgent medical evaluation. Because chest pain may indicate life-threatening cardiovascular disease, initial clinical assessment focuses on excluding cardiac causes, often resulting in extensive diagnostic testing and hospitalization. However, the majority of patients presenting with chest pain do not exhibit an underlying cardiac etiology and are diagnosed with non-cardiac chest pain (NCCP). NCCP is frequently attributable to esophageal disorders, including gastroesophageal reflux disease (GERD).

The esophagus is centrally located within the thorax, adjacent to the heart and major blood vessels. Irritation of esophageal tissues may therefore be perceived as cardiac pain. Proton pump inhibitors (PPIs), such as esomeprazole, provide limited benefit in many cases of unexplained chest pain and often require several days to weeks for therapeutic effect. A Shaped-Gel delivering a topical cooling agent to the upper esophagus may provide both diagnostic and therapeutic value by rapidly alleviating esophageal-origin discomfort and assisting in differentiation between cardiac and non-cardiac chest pain.

Reflux-related disorders arise when gastric contents migrate upward into the esophagus and pharynx. These conditions include GERD, laryngopharyngeal reflux (LPR), non-erosive reflux disease (NERD), NCCP, and functional dyspepsia. Many of these disorders are classified as functional, meaning that objective mucosal injury is absent in a substantial proportion of patients, despite significant symptoms. Functional reflux disorders account for the majority of heartburn cases.

Common symptoms of reflux disorders include heartburn, regurgitation, chest discomfort, early satiety, bloating, belching, nausea, and a sour or acidic taste in the throat. Heartburn is typically experienced as a substernal burning sensation that may radiate upward toward the neck and is often exacerbated by postures that promote reflux. In LPR, gastric acid and pepsin may reach the pharynx, larynx, and nasal passages, causing chronic cough, hoarseness, throat clearing, mucosal erythema, and edema.

Current management of reflux disorders relies primarily on acid-suppressive therapies, including PPIs, sometimes supplemented with antibiotics for H. pylori eradication, prokinetic agents, or other adjunctive therapies. There are, however, no therapies that directly address irritation of esophageal sensory nerve endings by acid and pepsin. A Shaped-Gel that delivers a cooling agent acting on TRPM8 receptors in the hypopharynx and upper esophagus offers a novel approach for alleviating reflux-associated discomfort and sensations of satiation.

### Mechanism of Action: Cooling Agent Targets on Esophageal Surfaces

The ion channel transient receptor potential melastatin-8 (TRPM8) is recognized as a physiological transducer of cooling sensations evoked by compounds such as menthol and icilin [Mckemy et al., Nature, 2002]. While TRPM8 activation is widely used as an in vitro screening endpoint for identifying candidate cooling agents, relatively little is known about how TRPM8 pharmacology predicts perceptual responses or therapeutic outcomes in humans. In particular, TRPM8 receptor potency, as measured by EC₅₀ values in heterologous expression systems, has limited ability to predict the quality, duration, tolerability, or therapeutic usefulness of cooling sensations when agents are applied to mucosal surfaces.

Indeed, Johnson et al., (2018) [Trigeminal Receptor Study of High-Intensity Cooling Agents. J, Agric. Food Chem., 66 (10), 2319-2323.] reported no meaningful correlation between TRPM8 EC₅₀ values and the perceived intensity of cooling produced by agents applied to the oral cavity of human volunteers. Similarly, although Zanone (U.S. Pat. No. 6,497,859) proposed that p-menthane carboxamides combined with menthyl acetate could alleviate gastrointestinal distress, subsequent human data demonstrated unexpected outcomes. Bancovín et al. reported that menthol infusion into the esophagus, while producing cooling sensations in healthy subjects, provoked substantial heartburn in patients with gastroesophageal reflux disease (GERD). These findings illustrate that cooling agents frequently exhibit complex, multivalent pharmacological effects that are not reliably predicted by TRP channel potency alone.

Structural studies using cryo-electron microscopy have identified binding sites for DIPA-1-9 on the TRPM8 receptor, with agonists acting at allosteric sites to facilitate ion channel opening. However, receptor binding and channel activation represent only one component of the overall sensory response. TRPM8-immunoreactive nerve fibers are present in the pharynx but are notably sparse or absent on the epiglottis. The hypopharynx extends from the hyoid bone to the inferior border of the cricoid cartilage and forms part of a continuous, funnel-shaped pharyngeal structure with an estimated mucosal surface area of approximately 10-15 cm².

Distribution studies demonstrate that TRPM8 expression is anatomically selective. Hondoh et al. showed that TRPM8 is scarce in the lower airway and that the neuronal cell bodies expressing TRPM8 are localized primarily within the jugular (JG) and petrosal (PG) ganglia, rather than the nodose ganglion (NG). By contrast, TRPA1-expressing neurons are found in all three ganglia. In the upper esophagus, TRPM8-expressing fibers arise from vagal and spinal afferents, whereas esophageal nociceptors responding to chemical and mechanical injury represent a partially distinct population and are not directly linked to TRPM8 signaling. These findings indicate that cooling and nociceptive signaling in the esophagus are anatomically proximate yet functionally separable.

Accordingly, the relevant physiological targets for the Shaped-Gel are not TRPM8 channels in isolation, but rather the receptive fields of sensory afferents associated with the glossopharyngeal (IX) nerve, the vagus (X) nerve, and spinal afferents innervating the upper third of the esophagus. Cooling signals and nociceptive signals generated within these receptive fields are transmitted by separate fiber populations and encoded in a modality-specific manner. Upon reaching central processing centers, these signals are integrated, and cooling input may suppress or modulate nociceptive perception.

As demonstrated in Study 1, reliance on TRPM8 EC₅₀ values as a primary indicator for candidate selection proved uninformative for predicting therapeutic efficacy in treating esophageal sensory discomfort. EC₅₀ confidence intervals frequently overlap across structurally distinct compounds, limiting discriminatory value. Moreover, TRPM8 EC₅₀ measurements provide no information regarding the qualitative character of cooling sensations, onset kinetics, duration of action, mucosal tolerability, or the likelihood of adverse perceptual effects such as irritation, burning, or unpleasant taste.

Once it became evident that TRPM8 receptor potency screening was of limited predictive value, alternative criteria for compound selection became essential. In the development of the present invention, human sensory data-derived from controlled exposure in human subjects-proved to be the only reliable indicator of clinically meaningful cooling, comfort, and duration of action. The inventor's prior experience and original testing in human subjects provided the foundation for identifying DIPA compounds capable of producing sustained, comfortable cooling in the hypopharynx and upper esophagus without provoking cold discomfort or heartburn.

The topographical proximity of cooling-sensitive afferents and nociceptive afferents in the upper esophagus further supports this approach. Although these fibers travel separately, their central integration allows cooling signals delivered at levels corresponding to the cricoid cartilage, jugular notch, and manubrium to suppress nociceptive input arising from esophageal irritation. Because the esophagus lies posterior to the trachea, cooling of esophageal sensory fields at these levels may additionally attenuate cough signals originating from adjacent airway structures.

TABLE 2
Neuronal Circuity of Afferents

| Afferent nerve | Brain Nuclei | Functions | Disorders |
|---|---|---|---|
| 5^{th} and 8^{th} | spinal trigeminal | mastication, | burning mouth |
| | nuclei | salivation, | syndrome |
| | | taste | |
| 9^{th} | para-trigeminal, | taste, thirst, | dysphagia |
| | dorsal nucleus of | swallowing | |
| | the vagus | | |
| 10^{th} (pharyngeal) | n. ambiguus, n. | cough | nausea, dysphagia |
| | tractus solitarius | | |
| 10^{th} (esophageal) | n. ambiguus, n. | thermosensory | nausea, reflux |
| | tractus solitarius | | discomfort, chest |
| | | | pain, cold discomfort |
| spinal nerves | | peristalsis | reflux discomfort, |
| | | | chest pain, cold |
| | | | discomfort |

### Swallowing the Shaped-Gel

Efficient swallowing mechanisms, exemplified by a pharyngeal transit time (PTT) of ≤1 second, reflect a highly optimized biological system designed to prevent entry of solid material into the airway while permitting rapid passage of food and liquids into the stomach. During swallowing, the epiglottis functions as a transient barrier, closing and reopening within seconds, while the upper esophageal sphincter similarly relaxes and recloses in a tightly coordinated sequence.

A swallowed bolus typically moves from the mouth to the esophagus within milliseconds. Pharyngeal transit time (PTT) is defined as the interval between arrival of the bolus tail at the fauces and complete passage through the upper esophageal sphincter and is commonly ≤1 second, as measured by videofluoroscopy. While this efficiency protects the airway, it presents a fundamental challenge for topical drug delivery to pharyngeal or esophageal surfaces. Solid particles are unsuitable for such delivery, as they may cause irritation and provoke coughing or choking reflexes. Consequently, agents intended for topical action in the throat must be delivered in liquid form or indirectly dissolved in saliva at the target site.

The coordinated movement of solids and liquids from the oral cavity to the stomach has been well described in the literature. During the oral phase, the bolus is positioned anteriorly with the tongue contacting the incisors while the palate compresses against the tongue to seal the oropharynx. Studies of upper esophageal sphincter mechanics demonstrate that sphincter opening increases with bolus volume from approximately 2 mL to 10 mL, but does not increase substantially with larger volumes. The sphincter opening is elliptical in shape, with a midline diameter of only approximately 5 mm and a total area increasing from roughly 120 mm² to about 240 mm² as bolus size increases. These dimensions indicate that temporary interception and delayed transit of a shaped, soluble object within the pharynx is feasible, particularly when the object dissolves and releases its contents prior to sphincter passage.

In practice, the Shaped-Gel delivers a cooling agent to the epiglottic valleculae, pyriform sinuses, upper esophageal sphincter, and the upper third of the esophagus. After deposition, the gelatinous matrix dissolves in saliva, typically resulting in a perceptible onset of cooling within approximately five minutes and symptom relief lasting at least one hour. With experience, individuals learn to use the formulation on an as-needed (pro re nata) basis. The rapid onset contributes to a sense of control over symptoms and may reduce anxiety and heightened vigilance commonly associated with throat and substernal discomfort.

A defining aspect of the present invention is the deliberate shaping of the Shaped-Gel to retard pharyngeal transit and promote temporary retention in the valleculae and pyriform sinuses above the upper esophageal sphincter. The Shaped-Gel is designed to dissolve at these sites and to disperse its active contents uniformly in saliva, facilitating contact with sensory receptors in the hypopharynx and upper esophagus. This strategy is counter to conventional pharmaceutical formulations, which are intentionally shaped to maximize swallowability and speed of transit to the stomach.

Criteria for an optimized Shaped-Gel include:

Mass: Approximately 0.5 to 1.0 g, preferably 0.6 to 0.8 g. A Shaped-Gel weighing≤0.5 g may transit too rapidly to allow sufficient dissolution, whereas units ≥1.0 g may be difficult for some individuals to swallow.

Liquid volume: Some individuals may require instruction to swallow the Shaped-Gel with little or no additional liquid or with a very small volume, such as ≤2 mL. Sipping from a water bottle (approximately 3-5 mL) is acceptable. Larger volumes of swallowed liquid may prematurely wash the Shaped-Gel into the lower esophagus.

Shape: The Shaped-Gel preferably has a generally flat geometry, wherein the shortest axis is approximately 5% to 45% of the longest axis. This geometry facilitates placement on the tongue, controlled entry into the pharynx, increased surface area, and enhanced retention within pharyngeal recesses.

Cut-outs or apertures: The Shaped-Gel may include one or more cut-outs or perforations to accelerate saliva penetration and dissolution. In toroidal embodiments, the central opening functions analogously to a “donut hole.” Rectilinear embodiments may incorporate punched apertures, and hemitoroid shapes are particularly effective for retention within pyriform sinuses. While other polygonal shapes have been evaluated, some may be perceived as unfamiliar or less acceptable to patients.

Material safety: The Shaped-Gel is formed from a gelatinous matrix that is soluble in saliva and water. Accordingly, even in the unlikely event of aspiration, the material readily dissolves and is absorbed, minimizing risk.

Composition and stability: The gelatinous matrix comprises a gelling agent, a plasticizer such as glycerol or sorbitol, and water, producing a product that is mechanically stable for storage and packaging, including blister packs. The formulation may be adapted for use by special populations, including children, elderly individuals, or those with impaired salivation or swallowing.

The Shaped-Gel may be administered on a fixed schedule or on an as-needed basis, depending on the clinical indication. As-needed administration allows users to exert voluntary control over pharyngeal and esophageal discomfort, supporting improved sleep quality, reduced anxiety, and enhanced quality of life. Fixed-interval dosing may be particularly beneficial in chronic conditions such as refractory cough, where reduction of neuronal hypersensitivity is a therapeutic goal.

### Study 1

### Selection of Cooling Agent: DIPA Compounds

The discovery relates to a particular compound within the series of compounds known as phosphine oxides (which have the following general formula), and more particularly, an example of the group known as di-alkyl-phosphinoyl-alkanes (herein referred to as “DAPA compounds”) (wherein each of R₁, R₂, and R³ is an alkyl group).

(O═)P R₁ R₂ R₃

And more specifically, to one particular 1-diisopropyl-phosphinoyl-alkane (DIPA), 1-Diisopropyl-phosphinoyl-nonane, referred to herein as “DIPA-1-9”. The DIPA compounds are colorless liquids with a density less than water. DIPA-1-9 is a liquid at room temperature, with a density of ~0.92 g/cm3 and a boiling point of 112-120° C. Note that DIPA-1-9 is achiral and does not have enantiomers.

TABLE 1
Chemical structure of DIPA-1-9

| | Chemical | Formula/ | |
|---|---|---|---|
| Code | Name | Weight | Chemical Structure |
| DIPA- 1-9 | 1-Diispropyl- phosphinoyl- nonane | C₁₅H₃₃OP 260.40 | 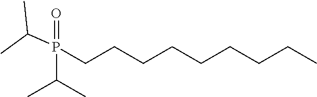 [SMILES: CCCCCCCCCP(=O)(C(C)C)C(C)C] |

The applicant has identified DIPA-1-9 as a prototype for the treatment of sensory discomfort arising from the membranes of the upper esophageal tract and surfaces. The applicant has reported on the efficacy of DIPA-1-9 for the membranes of the nasal cavity, for the transitional epithelium of the ocular surface (U.S. Pat. Nos. 9,642,868 and 9,895,382) and for the oropharyngeal surface. This is the first detailed report of the activities of DIPA-1-9 on the esophagus.

Chemical Synthesis DAPA compounds were prepared by the following general method: 100 mL (23.7 g, ~200 mmol) of sec-butylmagnesium chloride or bromide (isopropylmagnesium chloride or bromide) (obtained from Acros, as a 25% solution in tetrahydrofuran (THF)) was placed under nitrogen in a 500 mL flask (with a stir bar). Diethylphosphite solution in THF (from Aldrich, D99234; 8.25 g, 60.6 mmol in 50 mL) was added drop-wise. After approximately 30 min, the reaction mixture warmed up to boiling. The reaction mixture was stirred for an extra 30 min, followed by a drop-wise addition of the appropriate n-alkyl iodide solution in THF (from TCI; 60 mmol in 20 mL). In the case of DIPA-1-9, the n-alkyl halide was 1-iodononane. The reactive mixture was then stirred overnight at room temperature. The reaction mixture was diluted with water, transferred to a separatory funnel, acidified with acetic acid (~10 mL), and extracted twice with ether. The ether layer was washed with water and evaporated (RotaVap Buchi, bath temperature 40° C.). The light brown oil was distilled under high vacuum. The final products, verified by mass as determined by mass spectrometry, were clear liquids that were colourless or slightly pale yellow. The compounds prepared by these methods are shown in Table 4.

In the first set of data, the potency and in vitro effects of test compounds were evaluated on cloned hTRPM8 channel (encoded by the human TRPM8 gene, expressed in CHO cells) using a Fluo-8 calcium kit and a Fluorescence Imaging Plate Reader (FLIPR^{TETRA}™) instrument. The assays were by ChanTest Corporation (Cleveland, OH 44128, USA). Test solutions were in HEPES-buffered saline, 384-well plates, and placed into the FLIPR instrument (Molecular Devices Corporation, Union City, CA, USA). Four 4 to 8 concentrations were tested, with L-menthol as the positive control. The test cells were Chinese Hamster Ovary (CHO) cells stably transfected with human TRPM8 cDNAs. The concentration-response data were analyzed via FLIPR Control software and fitted to a Hill equation for the EC₅₀. The 95% Confidence Interval was from GraphPad Prism 6 software.

TABLE 2
Chemicals prepared and tested.

| Code | Chemical Name | Chemical Structure |
|---|---|---|
| DIPA-1-5 | 1-Di(isopropyl)- phosphinoyl-pentane | 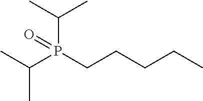 |
| DIPA-1-6 | 1-Di(isopropyl)- phosphinoyl-hexane | 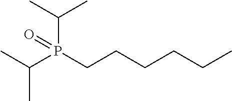 |
| DIPA-1-7 | 1-Di(isopropyl)- phosphinoyl-heptane | 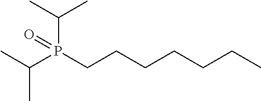 |
| DIPA-1-8 | 1-Di(isopropyl)- phosphinoyl-octane | 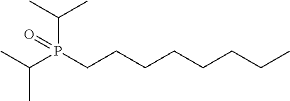 [SMILES: CCCCCCCCP(=O)(C(C)C)C(C)C] |
| DIPA-1-9 | 1-Di(isopropyl)- phosphinoyl-nonane | 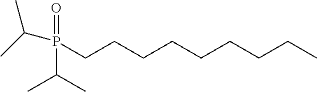 [SMILES: CCCCCCCCCP(=O)(C(C)C)C(C)C] |
| DAPA-2-4 | 1-Di(sec-butyl)- phosphinoyl-butane | 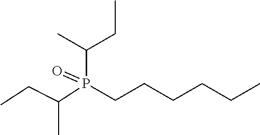 [SMILES: CCCCCCP(=O)(C(C)CC)C(C)CC] |
| DAPA-2-6 | 1-Di(sec-butyl)- phosphinoyl-hexane | 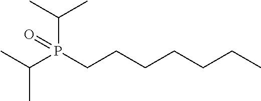 |
| DAPA-2-7 | 1-Di(sec-butyl)- phosphinoyl-heptane | 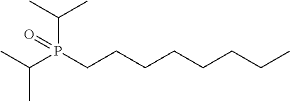 [SMILES: CCCCCCCCP(=O)(C(C)C)C(C)C] |
| DAPA-2-8 | 1-Di(sec-butyl)- phosphinoyl-octane | 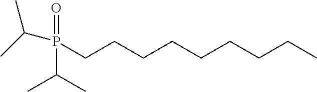 [SMILES: CCCCCCCCCP(=O)(C(C)C)C(C)C] |
| DAPA-2-6 | 1-Di(sec-butyl)- phosphinoyl- hexane | 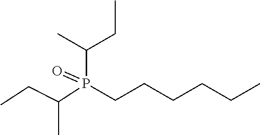 [SMILES: CCCCCCP(=O)(C(C)CC)C(C)CC] |

Table 5 summarizes the agonist activity in the TRPM8 receptor assay. All tested compounds showed full efficacy, i.e., at the highest tested concentration, there was ~100% stimulation of calcium entry, and the data fitted a sigmoidal dose-response curve. The EC₅₀ of the more potent sensory compounds DIPA-1-6 to 1-9 and DIPA-2-5 to 2-8 fell within a narrow range with overlapping 95% Confidence Intervals. No distinguishing features in the EC₅₀ predicted compounds with desired cooling properties in the esophageal tract.

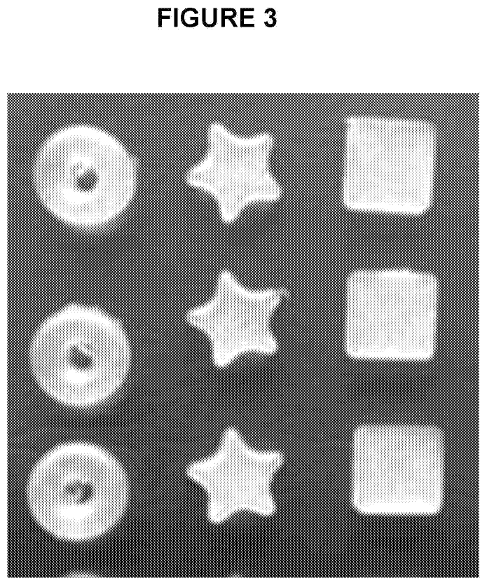

Selectivity is an index for gauging potential side effects of agonists. The selectivity of DIPA-1-9 on TRPA1, and TRPV1 was studied using transfected cells (human TRPV1 gene expressed in HEK293 cells and human TRPA1 expressed in CHO cells). DIPA-1-9 is specific for TRPM8 as shown in FIG. 3 of Yang et al. (A novel TRPM8 agonist relieves dry eye discomfort. BMC Ophthalmology (2017) 17:101). The applicant is a co-author of this publication. Selectivity is also seen with DIPA-1-7 and DIPA-1-8 (data in FIG. 1, Wei U.S. Pat. No. 9,956,232). The positive control reference compound was capsaicin (a known TRPV1 agonist) or mustard oil (TRPA1 agonist).

In summary, the relative potencies of these test series, as measured by the TRPM8 EC₅₀ [median effective dose], have limited predictive value for comparisons. The 95% Confidence Limits of many EC₅₀ overlap, and only analogs with at least a 5-fold difference in potency are distinguishable. The choice of an ideal ingredient requires obtaining the right degrees of coolness and avoiding icy cold sensations and adverse tastes. The duration of action is an important parameter. However, the EC₅₀ does not give information on the quality of the heat abstraction sensation, the likelihood of unpleasant taste, or the duration of the drug effect. Thus, the EC₅₀ is insufficient to define the desirable drug actions (access to and efficacy at TRPM8). Over interpretations of the EC₅₀ is misdirected.

In initial feasibility studies, three methods of applying a cooling agent to the throat were compared: namely, with a sprayer, by drops, or by a swallowed gel (Table 6). The dosimetry was such that a gel could deliver the largest dose without adverse events like bad taste or icy cold. The gel method gave a quantum jump in the duration of action, which was a major advantage. Cooling was felt at Adam's apple with all three delivery methods, but the gel gave the best results for the upper chest. Thus, the cooling agent in the gel gets onto the esophageal surface. Spray and drops were more likely to get into the mouth and affect the taste, a disadvantage not seen with the gel. The spray droplets could also get into the airways and trigger cough, a risk not seen with the drops or gel. The spray is a patient-familiar method of drug delivery, the drops less so, and swallowing the gel requires patient instruction. But patients soon become used to the gel, which has distinct advantages. If one weighs the evidence in toto, the gel is an excellent method for drug delivery to the pharynx and esophagus and is superior to spray or drops. Surprisingly, this method of gel delivery has not been much utilized for esophageal drug delivery.

TABLE 5
TRPM8 agonist activity of test compounds.

| | EC₅₀ | 95% Confidence | Relative Potency |
|---|---|---|---|
| Compound | (μM) | Interval | to L-menthol |
| Menthol | 3.8 | 2.5 to 5.6 | 1.0 |
| DIPA-1-5 | 5.6 | 4.4 to 7.2 | 0.7 |
| DIPA-1-6 | 2.4 | 1.5 to 4.0 | 1.6 |
| DIPA-1-7 | 0.7 | 0.5 to 1.0 | 5.4 |
| DIPA-1-8 | 0.7 | 0.5 to 1.0 | 5.4 |
| DIPA-1-9 | 0.9 | 0.4 TO 2.5 | 4.0 |
| DAPA-2-4 | 14.5 | 7 to 29 | 0.3 |
| DAPA-2-5 | 1.7 | 1.0 to 2.9 | 2.2 |
| DAPA-2-6 | 0.8 | 0.5 to 1.3 | 4.7 |
| DAPA-2-7 | 1.1 | 0.6 to 2.3 | 3.4 |
| DAPA-2-8 | 1.3 | 0.7 to 2.3 | 2.9 |

### Study 2

### Sensory Qualities of Compounds

Tests were on four volunteers, with 3 to 5 trials per substance. Compounds were prepared in Shaped-Gel at 5 mg/mL and administered ~0.8 mL per dose with a 2 mL plastic vial to the base of the tongue. The subjects asked to rate the sensations for cooling intensity, cold discomfort and adverse taste. DIPA-1-7 produced icy pain in the back of the throat. The compounds DIPA-1-7, DIPA-1-8, DAPA-2-6, DAPA-2-7 and 3,4-7 have adverse tastes described as “metallic” and “organic.” DIPA-1-9 Shaped-Gel produced a coolness and cold which was well-tolerated and the concentration could be increased to 15 mg/ml without objections: that is, there was no pain or discomfort.

TABLE 6
Delivery Systems to Surfaces of Pharynx-Esophagus

| Parameter | Sprayer | Drops | Gel | Comments |
|---|---|---|---|---|
| volume, mass | 0.4 mL | 0.4 mL | 0.3 to 0.8 g | — |
| concentration (mg) | 3 | 10 | 10 | — |
| total dose (mg) | ~1.2 | ~4 | ~3 to 8 | gel advantage |
| duration | ~10-15 | ~30-45 + | 60 + | gel advantage |
| mouth | +++ | ++ | 0 | gel advantage |
| upper throat | +++ | +++ | ++ | target jugular notch |
| chest | 0 | + | +++ | gel advantage |
| adverse taste | + | + | 0 | gel advantage |
| risk of entry to lung | + | 0 | 0 | — |
| familiarity of use | +++ | ++ | + | — |

When tested in the evening near sleep time, the perception of cooling in the throat was more pronounced presumably because there were fewer environmental cues for distraction. In these situations, cooling was perceived for a longer duration. The general sense of refreshment in the throat from DIPA-1-9 may persist for 3+ hours. Increasing the test concentration of DIPA-1-9 from 5 to 8 to 15 mg/mL in a Shaped-Gel did not produce icy cold or pain, indicating an adequate margin of safety.

### Study 3

Shaped-Gel having total weight of 0.4 to 1.2 g containing 0.4 to 1.5% by weight (5 mg/g to 15 mg/g) are prepared and tested, using the methods described by Thakkar (U.S. Pat. No. 9,913,873). The following examples illustrate the invention, but are not intended to limit the scope of the present invention.

A Shaped-Gel in accordance with the present invention was prepared with the following composition.

Each SHAPED-GEL: ~1 g; Cooling Agent: DIPA-1-9, 10 mg (1%); Gelatin: 120 mg (12%); Glycerol: 650 mg (65%); Water: 220 mg (22%).

Each SHAPED-GEL: ~0.8 g; Cooling Agent: DIPA-1-9, 8 mg (1%); Gelatin: 96 mg (12%); Glycerol: 520 mg (65%); Water: 176 mg (22%).

Each SHAPED-GEL: ~0.8 g; Cooling Agent: DIPA-1-7, DIPA-1-8, 8 mg (1%); Gelatin: 96 mg (12%); Glycerol: 520 mg (65%); Water: 176 mg (22%)

Each SHAPED-GEL: ~0.8 g; Cooling Agent: DAPA-2-5, DAPA-2-6, 8 mg (1%); Gelatin: 96 mg (12%); Glycerol: 520 mg (65%); Water: 176 mg (22%)

Each SHAPED-GEL: ~1.0 g; Cooling Agent: Ax-8 10 mg (1%), WS-5, WS-12, or WS-30; Ethanol: 50 mg (5%) g; Gelatin: 120 mg (12%); Glycerol: 650 mg (62%); Water: 220 mg (20%).

Shaped-Gels were prepared by these procedures. First step, the cooling agent was accurately weighed and place in a 60 mL plastic container. Then glycerol, water, and gelatin was added to the container and centrifuged for 2 min in a SpeedMixer (FlackTek, Inc.) at 3000 rpm. Then gelatin powder was added and the mixture centrifuged for 3 min. The container was warmed by placing it in a beaker containing hot water (80-85° C.) for 15 min. The container was then centrifuged for 5 min. The liquid mix was cooled and dispensed onto silicon molds with a plastic dropper, and placed in a refrigerator. After one hour, the gels were solidified, removed from the mold with a plastic tweezer, weighed and placed on wax paper and later stored in blister pockets. For some active ingredients 5% ethanol was added to increase solubility. Surprisingly, shaped-gels in blister packs retain their structure and cooling activity when stored at room temperature for at least 2 years.

In preliminary studies, the shape of the Shaped-Gel was varied. Square, rectangular, spherical, hemispherical, toroidal, and pentagram Shaped-Gels were prepared at sizes ranging from 0.4 to 1.2 g. A flat shape wherein the shortest diameter was about 20% of the longest axis was preferred because it could be placed flat on the back of the tongue. A Shaped-Gel shaped as a rectangular prism of 2.75 mm×15 mm×15 mm=0.63 cm³ and weighing ~0.6 g was acceptable to subjects. A toroid, much like a “LiveSaver” mint was also acceptable if the size was ~0.8 g.

To evaluate the effect of medicament geometry on pharyngeal residence, representative Shaped-Gel formulations having different three-dimensional shapes were assessed in a small observational study (Table 7). Five adult volunteers (four males and one female, ages 40 to 68 years) participated. Shaped-Gels having spherical, toroidal, square, and pentagonal geometries were prepared for testing. The Shaped-Gels consisted solely of the gelatin-based matrix and did not include an active cooling agent.

Each Shaped-Gel configuration was evaluated in multiple trials across at least four subjects. Following oral administration with approximately 5 ml of water, the duration of pharyngeal awareness of the Shaped-Gel was recorded as a proxy for residence time. For purposes of this assessment, pharyngeal awareness refers to a transient sensation associated with the presence of the medicament in the pharyngeal region prior to complete swallowing.

TABLE 7
Testing of Various Cooling Agents in a Shaped-Gel.

| | | Duraton | Adam’s | Jugular | | |
|---|---|---|---|---|---|---|
| Ingredient | Onset | min | Apple | Notch | Sternum | Discomfort |
| DIPA-1-9 | <3 | ~60 | ++ | ++ | ++ | 0 |
| DAPA-2-6 | <3 | 30 | +++ icy | ++ icy | ++ | yes |
| Ax-8 | 5 | 35 | ++ | + | + | − |
| WS-5 | ~3 | 25 | ++ | + | + | 0 |
| WS-12 | 5 | 20 | + | 0 | 0 | 0 |
| WS-30 | ~3 | 25 | ++ | + | + | 0 |

No appreciable pharyngeal awareness was reported with spherical Shaped Gels. In contrast, toroidal, square, and pentagonal Shaped Gels consistently produced measurable pharyngeal residence. The mean durations (±standard error of the mean; eight total trials per configuration) were approximately 42±4 seconds for toroidal shapes, 55±5 seconds for square shapes, and 35±4 seconds for pentagonal shapes. These observations demonstrate that non spherical geometries increase pharyngeal residence relative to spherical forms, thereby supporting the use of shaped medicaments to prolong local exposure prior to esophageal transit.

Test Panel: The volunteers for testing the Shaped-Gel containing a cooling agent were six subjects, four males and two females, aged 50 to 76 years. Each Shaped-Gel was tested for at least four trials in at least three subjects. The concentration of the cooling agent in the Shaped-Gel varied from 0.5 to 2%. The duration of observation for each trial usually lasted for not more than 1.5 hours. Subjects were instructed to swallow the Shaped-Gel with a sip of room temperature cold water. Cooling sensations were rated on a scale of 0, 1, 2, 3, at intervals of 3 min, 5 min, and then at 10 min intervals. If a cooling value exceeded 2, the subject noted whether the cooling was felt at the level of the Adam's apple (laryngeal prominence), jugular notch or upper chest. Ancillary effects were also recorded.

TABLE 8
Structures of p-Menthane compounds tested in Shaped-Gel.

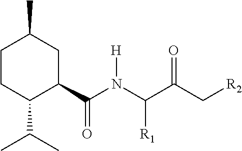

| | | | Philtrum | Eyelids | | Potency |
|---|---|---|---|---|---|---|
| | | | Skin | Surfaces | | relative to |
| Compounds | R₁ | R₂ | (min) | (min) | EC₅₀ μM | menthol |
| Menthol | | | | | 3.8 | 1.0 |
| | | | | | (2.5 to 5.6) | |
| Gly-OEt (WS- | H | Et | 24 | 15 | 0.36 | 10.6 |
| 5) | | | | | (0.30 to 0.75) | |
| Gly-OiPr-(Ax- | H | iPr | 27 | 300 | 0.50 | 7.6 |
| 8) | | | | | (0.27 to 0.85) | |
| D-Ala-OEt | Me | Et | 103 | 180 | 0.270 | 14.0 |
| | | | | | (0.17 to 0.42) | |
| D-Ala-OiPr | Me | iPr | 34 | 360 | 0.260 | 14.6 |
| | | | | | (0.16 to 0.41) | |

*These chemical structures are described in Wei, ET (2013). Ser. No. 13/065,971. [((1R,2S,5R) 2-isopropyl-5-methylcyclohexanecarbony)-amino]-acetic acid isopropyl ester and related compounds and their use in therapy. U.S. Pat. No. 8,426,463, Apr. 23, 2013.

These general effects were consistently observed: a rapid onset (≤3 min) of the sensation of coolness in the throat after application of DIPA-1-9 Shaped-Gel to the base of the tongue. The coolness spreads to the rest of throat and intensifies, as if a spoonful of rich ice cream had been swallowed. This cooling effect lasts for ≥45 min to up to 3 hr, and any prior discomfort in the throat is relieved. The cooling sensation can be used to facilitate mucus expectoration from the airways. Also relieved is the sense of suffocation when lying down to sleep in a subject that has dyspnea. Other cooling agent were also examined, as shown in the Table 8.

1. A method for treating symptoms of an esophageal disorder in a person in need thereof, comprising:
orally administering a solid medicament comprising a cooling agent in a gelatin glycerol water matrix,
the cooling agent consisting essentially of a 1 dialkyl phosphinoyl alkane,
wherein the medicament has a mass of from 0.5 to 1.0 grams per unit dose,
wherein the medicament is a non-spherical three dimensional object having three orthogonal axes, the shortest axis being from 5% to 30% of the length of the next longer axis,
wherein the medicament has a surface area to volume ratio greater than that of a sphere having the same mass,
and wherein the medicament is configured to be temporarily retained in the pharyngeal region for a period of from 30 to 60 or more seconds prior to passage into the esophagus.

2. The method of claim 1, wherein the medicament has a toroidal geometry, a hemi-toroidal geometry or a flattened polygonal geometry selected from a square, rectangle, pentagon, or hexagon.

3. The method of claim 1, wherein the gelatinous matrix dissolves upon contact with saliva to avoid airway obstruction.

4. The method of claim 1, wherein the cooling agent is present in an amount of less than 20 mg per unit dose.

5. The method of claim 1, wherein the cooling agent comprises at 0.4% (5 mg/g) to 1.5% (15 mg/g) by weight of the medicament.

6. The method of claim 1, wherein the medicament has a shape of a rectangular prism or a shape of a toroid.

7. The method of claim 1, wherein the medicament has one or more holes in the medicament matrix.

8. The method of claim 1, wherein the glycerol-gelatin matrix has a weight ratio of glycerol to gelatin that is at least two.

9. The method of claim 1, wherein the gelatin-glycerol-water matrix of the medicament pill is comprised of a gelatin of animal, vegetable, or marine origin.

10. The method of claim 1, wherein the cooling agent is 1-Diisopropyl-phosphinoyl-nonane (DIPA-1-9).

11. The method of claim 1, wherein a symptom of the disorder of the esophagus is caused by the disorder known as acid reflux, gastroesophageal reflux disease, laryngopharyngeal reflux, dyspepsia, or indigestion.

12. The method of claim 1, wherein a symptom of the disorder of the esophagus is selected from heartburn, epigastric pain, chest pain and sensation of fullness, bloat, belching, or a sour taste in the throat.

13. The method of claim 12, wherein the chest pain is non-cardiac chest pain.

14. A method for treating symptoms of an esophageal disorder in a person in need thereof, comprising:
orally administering a solid medicament comprising a cooling agent dispersed within a dissolvable gelatinous matrix,
wherein the medicament is a non-spherical, three-dimensional object having dimensions selected to delay pharyngeal transit during swallowing,
wherein the medicament has an increased surface area-to-volume ratio relative to a sphere of equal mass,
wherein the medicament is configured to be temporarily retained within the pharyngeal region, including at least one of the valleculae or pyriform sinuses, for a sufficient period of time to permit dissolution of the gelatinous matrix in saliva,
wherein dissolution of the gelatinous matrix releases the cooling agent onto mucosal surfaces of the hypopharynx and upper esophagus,
and wherein the cooling agent is selected from the group consisting of:
(a) a 1-dialkyl-phosphinoyl-alkane,
(b) a p-menthanecarboxamide, and
(c) a cooling compound that, upon topical contact with pharyngeal or upper esophageal mucosa, produces a cooling sensation perceptible to a human subject.

15. The method of claim 14, wherein the 1-dialkyl-phosphinoyl-alkane is 1-Diisopropyl-phosphinoyl-nonane.

16. The method of claim 14, wherein the p-menthanecarboxamide cooling agent is ((1R,2S,5R) 2-isopropyl-5-methylcyclohexanecarbony)-amino]-acetic acid isopropyl ester.

17. The method of claim 14, wherein a symptom of the disorder of the esophagus is caused by the disorder known as acid reflux, gastroesophageal reflux disease, laryngopharyngeal reflux, dyspepsia, or indigestion.

18. The method of claim 14, wherein the cooling agent is selected based on producing a cooling sensation in human subjects without inducing esophageal irritation or heartburn.

19. The method of claim 14, wherein the non-spherical geometry and the cooling agent are co-selected to cooperatively increase residence time and produce sustained cooling of the hypopharynx and upper esophagus.

20. A method for treating esophageal sensory discomfort in a person in need thereof, comprising delivering a cooling agent to the hypopharynx and upper esophagus by temporarily retaining a non-spherical, saliva-soluble gelatinous medicament within the pharynx during swallowing.
